(19) United States
(12) Patent Application Publication
(10) Pub. No.: US 2025/0127766 A1
(43) Pub. Date: Apr. 24, 2025

(54) METHODS AND COMPOSITIONS FOR TREATING CDKL5 DISORDER USING MONOAMINE OXIDASE INHIBITORS

(71) Applicant: Jainu Jogani, Fremont, CA (US)

(72) Inventor: Jainu Jogani, Fremont, CA (US)

(21) Appl. No.: 18/921,547

(22) Filed: Oct. 21, 2024

Related U.S. Application Data
(60) Provisional application No. 63/544,971, filed on Oct. 20, 2023.

(51) Int. Cl. A61K 31/437; A61K 31/19; A61K 31/4045; A61K 31/4166; A61K 31/53; A61K 31/5513; A61K 31/7048; A61P 25/00; A61P 25/08

(52) U.S. Cl. CPC A61K 31/437; A61K 31/19; A61K 31/4045; A61K 31/4166; A61K 31/53; A61K 31/5513; A61K 31/7048; A61P 25/00; A61P 25/08

(57) ABSTRACT

The present disclosure describes methods and compositions for treating CDKL5 deficiency disorder (CDD), a severe neurodevelopmental disorder characterized by early-onset, treatment-resistant epilepsy and global developmental impairment. The invention describes the use of monoamine oxidase inhibitors (MAO-Is), particularly MAO-A inhibitors such as harmine, for reducing convulsive seizure frequency in patients suffering from CDD. The MAO-Is may be administered alone or in combination with one or more anti-epileptic drugs (AEDs). The disclosed methods further include treatment strategies targeting CDKL5 paralogues to modulate neuronal signaling pathways and synaptic function. In vivo and in vitro studies demonstrate the efficacy of MAO-Is in upregulating CDKL5 protein expression and restoring synaptic homeostasis, offering a novel therapeutic approach for treating CDKL5-related epilepsy and other symptoms associated with the disorder.

### CROSS-REFERENCE

This application claims priority to U.S. provisional application no. 63/544,971, filed on Oct. 20, 2023, the contents of which are hereby incorporated by reference in its entirety.

### TECHNICAL FIELD

The present disclosure relates to methods and compositions for the treatment of neurological disorders, specifically CDKL5 deficiency disorder (CDD), with small molecules. More particularly, the disclosure pertains to the use of monoamine oxidase inhibitors (MAO-Is) for reducing convulsive seizure frequency in patients with treatment-resistant epilepsy associated with CDD, and for modulating synaptic function and neuronal signaling.

### BACKGROUND

CDKL5 deficiency disorder (CDD) is a rare and severe neurodevelopmental disorder caused by mutations in the CDKL5 gene, which is involved in brain development and synaptic function. CDD is characterized by early-onset, treatment-resistant epilepsy, profound intellectual disability, and significant motor and developmental impairments. Current treatment options, primarily aimed at managing seizures, offer limited efficacy and are not disease-modifying.

Monoamine oxidase inhibitors (MAO-Is) are traditionally used to treat neurological conditions such as depression and Parkinson's disease by increasing neurotransmitter levels in the brain. However, their potential for treating epilepsy, particularly in the context of CDD, has not been fully explored. This invention addresses the unmet need for an effective therapeutic approach targeting the underlying mechanisms of CDD.

### SUMMARY

The present disclosure relates to methods for treating CDKL5 deficiency disorder (CDD) in subjects by administering therapeutically effective amounts of monoamine oxidase inhibitors (MAO-Is), which have been shown to reduce convulsive seizure frequency and modulate neuronal signaling pathways. Specifically, the disclosure includes the discovery that MAO-Is, such as harmine, upregulate CDKL5 paralogues and restore synaptic function, providing a novel therapeutic strategy for CDD. The method includes administering a MAO-I, which may selectively inhibit either monoamine oxidase-A or monoamine oxidase-B, depending on the clinical scenario.

In one embodiment, harmine or its pharmaceutically acceptable salts, solvates, or derivatives are administered to the subject at dosage ranges of 0.5 to 10 mg/kg per day, particularly for pediatric patients. This administration may be conducted via various routes, including oral, intravenous, or transdermal application. In another embodiment, the method may further include administering one or more anti-epileptic drugs (AEDs) in combination with the MAO-I to enhance therapeutic efficacy. The AEDs can be selected from a group consisting of valproic acid, lamotrigine, topiramate, clonazepam, and phenytoin, among others.

Additionally, the disclosure encompasses methods for monitoring the subject's seizure activity and adjusting the dosage of the MAO-I based on the subject's clinical response. Another aspect of the invention includes increasing the phosphorylation of microtubule-associated protein RP/EB family member 2 (EB2) at Serine 222, which enhances synaptic function, thereby offering a targeted therapeutic effect.

In another embodiment, harmine or liquiritin is administered at a dosage of 1 to 10 mg/kg per day, and may be used in combination with valproic acid, melatonin, or other therapeutic agents. The combination can include continuous EEG monitoring to assess seizure activity and cognitive function improvement. The invention also provides for the co-administration of harmine or liquiritin with anticonvulsant agents, timed sequentially to reduce potential drug interactions.

### BRIEF DESCRIPTION OF THE DRAWINGS

The accompanying drawings are provided solely for illustrative and exemplary purposes and are not intended to limit the scope of the invention in any way. The invention is defined solely by the appended claims, which set forth the full scope of the invention. The drawings are merely illustrative examples to assist in understanding certain embodiments and implementations of the invention and should not be construed as limiting the invention to the specific structures or methods shown. Any variations or modifications within the scope of the claims are to be considered as part of the invention.

FIG. 1 shows a five-number summary graph for the CDKL5 protein expression in 5 wild-type mice (“WT”), 5 heterogeneous mice, and 5 heterogenous (Het) mice treated with liquiritin (RB 8437).

### DETAILED DESCRIPTION

The present disclosure provides methods and compositions for treating CDKL5 deficiency disorder (CDD) by utilizing monoamine oxidase inhibitors (MAO-Is), particularly those that selectively inhibit MAO-A, to reduce convulsive seizure frequency and modulate neuronal signaling pathways. The present disclosure is based on the discovery that MAO-Is, including but not limited to harmine, can upregulate CDKL5 paralogues and restore synaptic function, thereby addressing both the neurological symptoms and underlying molecular deficits associated with CDD. The methods described herein involve administering MAO-Is alone or in combination with one or more anti-epileptic drugs (AEDs), providing a novel therapeutic approach for patients suffering from treatment-resistant epilepsy related to CDKL5 mutations. Further, the disclosure encompasses various formulations, dosages, and administration routes to optimize therapeutic outcomes for different patient populations. The detailed description that follows outlines specific embodiments of the invention, experimental data supporting its efficacy, and additional features that contribute to the therapeutic potential of MAO-Is in managing CDKL5 deficiency disorder.

The present disclosure specifically relates to the use of MAO-Is for the reduction of total convulsive seizure frequency in the treatment of treatment-resistant epilepsy (TRE) caused by CDKL5 disorder. This method is applicable to a broad range of subjects in need, including human infants, children, and adults. The MAO-Is can be employed either as standalone agents or in combination with other AEDs to maximize therapeutic efficacy.

In one embodiment, the MAO-I is administered together with one or more AEDs. These AEDs may include, but are not limited to, narrow-spectrum AEDs such as phenytoin, carbamazepine, pregabalin, oxcarbazepine, and lacosamide, or broad-spectrum AEDs such as valproic acid, lamotrigine, topiramate, clonazepam, rufinamide, and sodium valproate. The combination of MAO-Is with these AEDs offers a comprehensive approach to managing seizures in patients with CDKL5 deficiency, particularly those who have not responded to conventional monotherapies.

Additionally or alternatively, the MAO-I may be formulated for administration separately, sequentially, or simultaneously with one or more AEDs. In certain embodiments, the combination of MAO-Is and AEDs may be provided in a single dosage form for ease of administration. Where the MAO-I is administered separately, sequentially, or simultaneously, it may be provided as part of a therapeutic kit, along with instructions for proper dosing and administration. Such flexibility in formulation allows for personalized treatment regimens based on the patient's age, seizure frequency, and coexisting conditions.

In one implementation, the MAO-I may be in the form of a pharmaceutical composition, which could include the MAO-I itself or in combination with one or more AEDs. The MAO-I may comprise harmine or its derivatives, which have been shown to effectively restore synaptic deficits and regulate neuronal excitability in CDKL5 deficiency disorder. The pharmaceutical composition may be tailored to provide therapeutically effective doses, ensuring that the patient receives optimal benefits in terms of seizure control and neurological function.

Pursuant to another aspect of the invention, there is provided a method for the treatment of CDKL5 disorder by administering a therapeutically effective dose of a pharmaceutical composition to a subject. This pharmaceutical composition comprises MAO-Is, optionally combined with one or more AEDs, and may include harmine or its derivatives. The flexibility in treatment regimens allows for the customization of dosage and administration routes to suit the specific needs of individual patients, ensuring that therapeutic objectives such as seizure reduction, improvement in cognitive function, and overall neurological stabilization are achieved.

The invention also encompasses various formulations, dosages, and routes of administration, such as oral, intravenous, or transdermal, depending on the patient's condition and therapeutic requirements. The combination of MAO-Is with existing AEDs or as a monotherapy offers a new and promising therapeutic avenue for managing the complex and treatment-resistant nature of CDKL5-related epilepsy, with the potential to improve the quality of life for affected individuals.

In addition to their established use in treating depression, neurodegenerative disorders, and epilepsy, monoamine oxidase inhibitors (MAO-Is) have shown potential in treating other conditions, such as posttraumatic stress disorder (PTSD) and Fragile-X syndrome. Emerging research suggests that MAO-Is may also be effective in treating female epilepsy associated with mutations in the protocadherin 19 gene (PCDH19), a rare form of epilepsy. Moreover, MAO-Is have been identified as a potential therapeutic option for managing sleep disruptions in patients with CDKL5 deficiency disorder, offering a method to alleviate one of the many neurological symptoms seen in these patients by modulating neurotransmitter activity.

### Cyclin-Dependent Kinase-Like 5 (CDKL5)

CDKL5 proteins mediate neurotransmitter release, reception, and regulation. CDKL5 is a gene that encodes a protein kinase important for normal brain development and functioning. CDKL5 disorder thus impacts neurotransmitter systems in the brain, leading to changes in neurotransmitter levels and functioning. Here's how neurotransmitters may be impacted by CDKL5 deficiency:

CDKL5 deficiency is a severe neurological disorder that affects normal brain development and function by leading to synaptic dysfunction and impaired neuronal connectivity. Synapses are the specialized structures where neurons communicate with each other through chemical and electrical signals. Proper synaptic function is critical for neuronal plasticity, learning, and memory, all of which are severely affected in individuals with CDKL5 deficiency disorder (CDD). CDKL5 is a protein kinase, an enzyme that adds phosphate groups to other proteins, thereby regulating their activity. In the case of synaptic function, CDKL5 plays a key role in ensuring proper signal transmission and synaptic integrity by modulating the phosphorylation states of various proteins involved in synaptic signaling pathways.

One of the main consequences of CDKL5 deficiency is the loss of regulation of a protein called DYRK1A (Dual-specificity tyrosine-phosphorylation-regulated kinase 1A), which is also involved in synaptic regulation. DYRK1A is a kinase that adds phosphate groups to other proteins, thereby regulating their activity. Under normal conditions, CDKL5 inhibits the excessive activity of DYRK1A by phosphorylating it. This phosphorylation of DYRK1A by CDKL5 serves to inhibit DYRK1A's activity, preventing it from over-phosphorylating its downstream targets. Over-phosphorylation of proteins by DYRK1A can lead to disruption of synaptic function, improper neuronal signaling, and ultimately the synaptic impairments observed in CDKL5-deficient individuals.

Without functional CDKL5, DYRK1A remains unregulated and becomes overactive, leading to excessive phosphorylation of its substrates, which are critical proteins involved in maintaining synaptic integrity and neuronal connectivity. This dysregulation of DYRK1A results in the breakdown of synaptic connections and impaired communication between neurons. The loss of synaptic plasticity and the inability of neurons to properly transmit signals contribute to the neurological symptoms seen in CDD, such as seizures, developmental delays, and intellectual disabilities.

### Monoamine Oxidase (MAO)

MAO is an enzyme responsible for breaking down neurotransmitters such as serotonin, dopamine, and norepinephrine, which play crucial roles in mood regulation, neuronal signaling, and various physiological functions. There are two types of MAO: MAO-A and MAO-B, each with distinct tissue distribution and function.

MAO-A is primarily found in the liver and intestines and is responsible for the breakdown of neurotransmitters like serotonin, norepinephrine, and melatonin. Because of its location and function, MAO-A inhibitors are often used to treat conditions such as depression and anxiety. However, MAO-A inhibitors are more likely to interact with other medications, including antidepressants, antipsychotics, and blood pressure medications, leading to potential drug interactions and side effects such as headaches, dizziness, and nausea. These interactions are particularly important when considering the risk of hypertensive crises associated with dietary intake of tyramine, although newer, reversible inhibitors have reduced these risks.

MAO-B, on the other hand, is primarily found in the brain and is responsible for breaking down neurotransmitters like dopamine and certain trace amines. Given its presence in the brain, MAO-B inhibitors are particularly relevant in treating neurological conditions such as Parkinson's disease, Alzheimer's disease, and epilepsy. For example, safinamide, an MAO-B inhibitor, has demonstrated anticonvulsant properties in animal models, where it has been shown to suppress seizures and provide neuroprotective effects, preventing damage to neurons.

### Neurotransmitters

Neurotransmitters play critical roles in regulating a variety of physiological and neurological processes, including mood, motor control, and cognitive functions. In the context of CDKL5 deficiency disorder (CDD), several neurotransmitter systems are disrupted, contributing to the neurological and behavioral symptoms commonly seen in affected individuals.

Serotonin is a key neurotransmitter involved in mood regulation, sleep, and other essential functions. In individuals with CDKL5 deficiency, alterations in serotonin signaling are common and may contribute to mood disturbances, sleep disorders, and behavioral issues. Research has shown that increasing serotonergic activity can provide therapeutic benefits to children with CDD. Monoamine oxidase inhibitors (MAOIs), by inhibiting the breakdown of serotonin, increase its levels in the brain, potentially mitigating some of the serotonin-related symptoms in these patients.

The endocannabinoid system, which includes neurotransmitters such as anandamide, regulates a range of physiological functions, including pain perception, mood, and appetite. Dysregulation of this system has been implicated in increased pain sensitivity and mood disturbances in individuals with CDKL5 deficiency. Serotonin and the endocannabinoid system are closely interconnected, with serotonin levels influencing endocannabinoid signaling. By increasing serotonin levels through MAOIs, there may be indirect modulation of the endocannabinoid system, restoring balance and potentially improving both mood and pain regulation.

Dopamine, another important neurotransmitter, is associated with reward, motivation, and motor control. Imbalances in dopamine levels are observed in CDKL5 deficiency, potentially contributing to motor problems and mood-related symptoms. MAO-B, an enzyme predominantly found in the brain, catalyzes the breakdown of dopamine into its metabolites, including 3,4-dihydroxyphenylacetaldehyde (DOPAL) and 3,4-dihydroxyphenylacetic acid (DOPAC). By inhibiting MAO-B, dopamine levels can be increased, potentially alleviating some of the motor and mood disturbances seen in CDD.

Norepinephrine plays a crucial role in arousal, attention, and the body's “fight or flight” response. In individuals with CDKL5 deficiency, dysregulation of norepinephrine signaling may affect attention and arousal levels. MAOIs, by inhibiting the breakdown of norepinephrine, increase its levels in the brain, which may help improve focus, attention, and overall arousal in individuals with CDD.

Finally, glutamate, the brain's primary excitatory neurotransmitter, is essential for synaptic plasticity and learning. Imbalances in glutamate signaling can lead to cognitive impairments, as seen in CDKL5 deficiency disorder. Alterations in glutamate transmission contribute to the neurological symptoms in CDD, and restoring balance to glutamate levels may help improve cognitive function in affected individuals.

### Monoamine Oxidase Inhibitors (MAO-I)

Monoamine Oxidase Inhibitors (MAO-Is) are a class of compounds that function by inhibiting the activity of monoamine oxidase (MAO) enzymes, which are responsible for the breakdown of several key neurotransmitters in the brain, including serotonin, dopamine, norepinephrine, and others. By inhibiting these enzymes, MAO-Is increase the levels of these neurotransmitters, thereby enhancing their availability and activity in the brain. This action results in a range of therapeutic effects, such as improving mood, reducing anxiety, and slowing the progression of neurological diseases like Parkinson's disease and Alzheimer's disease.

There are two types of MAO enzymes: MAO-A and MAO-B. MAO-A inhibitors primarily affect the breakdown of serotonin and norepinephrine and are often associated with the treatment of depression and anxiety disorders. However, MAO-A inhibitors tend to have more potential for drug interactions and side effects, including headaches, dizziness, and nausea. MAO-B inhibitors, in contrast, predominantly affect dopamine and are more commonly used in the treatment of neurodegenerative diseases such as Parkinson's disease. While MAO-B inhibitors generally have fewer side effects than their MAO-A counterparts, they can still cause similar adverse effects due to their interaction with neurotransmitter systems.

In addition to affecting neurotransmitters like dopamine, MAO-B plays a role in the regulation of glutamate, the brain's primary excitatory neurotransmitter. MAO-B indirectly modulates glutamate signaling and helps to balance the levels of gamma-aminobutyric acid (GABA), the brain's main inhibitory neurotransmitter. This balance between excitatory and inhibitory neurotransmission is crucial for normal brain function. In CDKL5 deficiency disorder (CDD), this balance is disrupted, leading to seizures and cognitive impairments. By inhibiting MAO-B, MAO-Is help regulate GABAergic signaling, promoting inhibitory signals that may reduce excitotoxicity—a process where excessive glutamate activity can lead to neuronal damage or death.

The neuroprotective effects of MAO-Is are related to their ability to regulate neurotransmitter systems and prevent excitotoxicity. For example, MAO-B can metabolize glutamate into GABA, helping to prevent overexcitation of neurons. Harmine and other MAO-Is have been shown to increase GABA receptor activity and inhibit GABA transporters, which further enhances the calming, inhibitory effects of GABA on the brain. This modulation of GABA receptors by MAO-Is has been demonstrated in both in vitro and in vivo studies. For instance, in vitro studies have shown that harmine increases GABA receptor activity, while in vivo studies in animal models, such as rats and mice, have demonstrated that MAO-Is reduce anxiety and increase sleep duration, both of which are linked to enhanced GABA signaling.

Additionally, MAO-Is have been investigated for their neuroprotective properties. In various studies, MAO-Is have been shown to protect neurons from damage and promote neurogenesis, the formation of new neurons. This is particularly important in neurodevelopmental disorders like CDKL5 deficiency, where disrupted neurotransmission leads to cognitive impairments and seizure activity. The GABAergic signaling promoted by MAO-Is may contribute to these neuroprotective effects by reducing neuronal stress and preventing excitotoxicity, ultimately improving overall brain function and providing therapeutic benefits in conditions like CDKL5 deficiency disorder.

MAO-Is are categorized based on their selectivity for MAO-A or MAO-B, with several compounds targeting either enzyme specifically. MAO-A inhibitors include harmine, a selective MAO-A inhibitor, as well as moclobemide, a reversible MAO-A inhibitor commonly used in treating depression. Other MAO-A inhibitors are clorgyline, pirlindole, toloxatone, and befloxatone, all of which inhibit MAO-A activity and are primarily used for their antidepressant effects.

MAO-B inhibitors, on the other hand, are often used in the treatment of Parkinson's disease due to their role in preventing the breakdown of dopamine in the brain. Examples include selegiline and rasagiline, both of which are irreversible inhibitors of MAO-B. Another key MAO-B inhibitor is safinamide, which is a reversible inhibitor also used in Parkinson's therapy. In some cases, phenelzine and isocarboxazid, which are non-selective MAO inhibitors, demonstrate a preference for MAO-B at lower doses.

Finally, there are non-selective MAO inhibitors that inhibit both MAO-A and MAO-B. These include phenelzine, tranylcypromine, and isocarboxazid, all of which are irreversible inhibitors used primarily for treating mood disorders. Linezolid, though primarily an antibiotic, also exhibits reversible inhibition of both MAO-A and MAO-B. The choice of MAO inhibitor depends on the therapeutic context, whether for treating depression, anxiety disorders, or neurological conditions such as Parkinson's disease.

Inasfar as dosing is concerned, Safinamide may use a typical human doses ranging from 50 to 100 mg per day. Given its demonstrated anticonvulsant activity in animal models, it is plausible that similar dosing could be effective for the treatment of CDD. However, clinical testing is still needed to confirm its efficacy and safety for this specific use.

When MAO-Is are used in combination with anti-epileptic drugs (AEDs) such as valproic acid, lamotrigine, or clonazepam, it is essential to administer the AEDs at their standard therapeutic doses. These can range from 500 mg/day for valproic acid to 5-20 mg/day for clonazepam, depending on the specific AED being used. In these combination therapies, MAO-A inhibitors should be titrated carefully, starting with lower doses such as 1-2 mg/day in children and potentially up to 10 mg/day in adults, ensuring that interactions with AEDs are minimized.

The dosing regimens for another MAO-I, phenelzine, varies depending on the patient's age, condition, and response to treatment. For adults with depression, typical dosing ranges from 15 mg orally every other day to 30 mg orally three times daily. Initial treatment commonly begins at 15 mg orally three times per day, with the dose gradually increasing to 60 to 90 mg daily, divided into three or four doses, depending on the patient's response. The goal is to establish the minimal effective dose, with a maximum limit of 90 mg per day.

In elderly patients, the therapeutic dose typically ranges from 15 to 60 mg daily, divided into three or four doses. Starting doses are lower, often beginning at 7.5 mg orally per day, with increments of 7.5 to 15 mg every three to four days as tolerated. For pediatric patients, particularly those with selective mutism (an unlabeled indication), dosing ranges from 30 to 60 mg per day in divided doses.

Phenelzine is contraindicated in patients with severe renal insufficiency, and its use in patients undergoing peritoneal or hemodialysis remains undefined. Additionally, it should not be administered to patients with a history of hepatic disease or elevated liver function tests (LFTs). Clinicians must provide patients with an FDA-approved medication guide when dispensing phenelzine, particularly in outpatient settings where the patient will take the medication without direct supervision. This is especially crucial given the FDA Box Warning regarding the increased risk of suicidal thoughts and behaviors in children, adolescents, and young adults (ages 18 to 24) who are being treated for major depressive disorder or other psychiatric conditions. Phenelzine is not FDA-approved for treating depression in children under 16 years of age.

The mean elimination half-life of a single 30 mg dose of phenelzine is approximately 11 to 12 hours, though multiple-dose pharmacokinetics have not been thoroughly studied in humans. The onset of antidepressant effects typically takes two to three weeks. Upon discontinuation, the dose should be tapered slowly to minimize withdrawal symptoms. If transitioning to another antidepressant, three to four weeks should be allowed between discontinuation of phenelzine and initiation of the new therapy. In cases where concurrent administration of phenelzine with another antidepressant is necessary, or if starting phenelzine within 10 days of discontinuing a different antidepressant, the prescriber must counsel the patient on the risks of adverse drug interactions.

Monoamine oxidase inhibitors (MAO-Is) can be administered either orally or intravenously, depending on the formulation and the specific needs of the patient. Oral administration typically requires daily dosing, while intravenous formulations may allow for quicker adjustments based on pharmacokinetic requirements. For pediatric patients, oral suspensions or gradual-release capsules are preferable to maintain consistent therapeutic levels and minimize fluctuations in drug concentration.

Formulations can be optimized for specific routes of administration to enhance therapeutic efficacy and patient compliance. For oral delivery, pharmaceutical compositions are formulated with pharmaceutically acceptable carriers, including tablets, pills, capsules, solutions, suspensions, sustained-release formulations, powders, liquids, or gels, which are suitable for oral ingestion. For alternative delivery methods, compositions may be administered transdermally. Transdermal patches are beneficial because both MAO-Is and NMDA receptor antagonists exhibit high skin permeability, avoiding the adverse events and pharmacokinetic variability associated with first-pass metabolism.

Additionally, pharmaceutical compositions may be delivered via inhalation or insufflation using aerosol sprays, nebulizers, or dry powder inhalers. Suitable propellants for nebulization include dichlorodifluoromethane, trichlorofluoromethane, dichlorotetrafluoroethane, and carbon dioxide. Dosage may be regulated via a valve to control the amount delivered in pressurized aerosols. Compositions for inhalation or insufflation include solutions and suspensions in pharmaceutically acceptable aqueous or organic solvents, as well as powders, and may contain excipients as described above. These compositions may be administered via the oral, intranasal, or respiratory route for local or systemic effects. In some embodiments, the composition is delivered intranasally to the cribriform plate, facilitating transfer into the central nervous system (CNS) via the olfactory passages, which can enhance CNS dosing while reducing systemic exposure and associated toxicities. Devices for this route of administration may be utilized to deliver formulations directly to the CNS.

Additional formulations suitable for other modes of administration include rectal capsules or suppositories. These suppositories may be formed using traditional binders and carriers, such as polyalkylene glycols or triglycerides, with the active ingredient constituting 0.5% to 10% of the formulation, preferably 1%-2%. Long-term delivery formulations are also possible, such as continuous-release vessels composed of biocompatible materials like titanium, enabling delivery for extended periods (e.g., 30, 60, 90, 180 days, or up to one year). These formulations are particularly useful for chronic conditions, improving patient compliance, and enhancing the stability of the pharmaceutical compositions.

The dosage of MAO-Is should be adjusted according to the patient's treatment response and the presence of any adverse effects, such as headaches, dizziness, or nausea, which are more commonly observed with MAO-A inhibitors. Continuous neurological monitoring, along with regular EEG assessments, will be essential to ensure that the optimal therapeutic dose is being administered to each individual patient, ensuring both efficacy and safety in long-term management.

The present invention may also include the use of NMDA receptor antagonists, such as aminoadamantane compounds, in combination with monoamine oxidase inhibitors (MAO-Is) for treating CDKL5 deficiency disorder. Suitable aminoadamantane compounds include, but are not limited to, memantine, amantadine, rimantadine, and their pharmaceutically acceptable salts. These compounds have been well-documented for their ability to modulate NMDA receptor activity and have been described in numerous U.S. patents, which are incorporated herein by reference. In this context, the combination of NMDA receptor antagonists and MAO-Is may provide a synergistic effect, particularly in neurological disorders like CDKL5 deficiency, where synaptic dysfunction and excitotoxicity are prevalent.

The NMDA receptor antagonist may be administered in combination with MAO-Is, with doses ranging from 1 to 80 mg/day for memantine, 25 to 500 mg/day for amantadine, or 1 to 5000 mg/day for dextromethorphan, depending on the specific agent and patient profile. Pediatric doses would generally be lower. Additionally, the formulation may be designed to maintain sustained release, with a Cmax/Cmean ratio of approximately 2 or less for at least 2 to 8 hours post-administration. This controlled release may enhance therapeutic outcomes while minimizing potential side effects.

Finally, the components of the therapeutic combination may be provided as a kit, with one or more containers containing the NMDA receptor antagonist and the MAO inhibitor or GAPDHai, either mixed or provided separately. The kit may also include instructions for use, offering a therapeutically effective dose of agents for treating dementia-related conditions.

Derivatives of monoamine oxidase inhibitors (MAO-Is) represent a critical area of development in tailoring treatments for neurological and psychiatric disorders while mitigating associated risks, particularly those related to food-drug interactions. As MAO has two isoforms, MAO-A and MAO-B, each with distinct physiological roles, the development of selective derivatives has allowed for more targeted therapeutic approaches.

At the risk of being redundant, MAO-A derivatives primarily focus on modulating the metabolism of circulating monoamines, such as epinephrine, norepinephrine, and dopamine. These derivatives are especially relevant in treating conditions like depression, where precise control of neurotransmitter levels is essential. By selectively inhibiting MAO-A, these derivatives avoid inhibiting MAO-A in the gastrointestinal (GI) tract, reducing the risk of tyramine-induced hypertensive crises, a known risk of non-selective MAO inhibitors. However, selective MAO-A derivatives remain less commonly used due to the availability of other antidepressant therapies, though they are still explored for their unique benefits in treatment-resistant depression.

On the other hand, MAO-B derivatives are more extensively studied for their role in neurodegenerative disorders, such as Parkinson's disease and Alzheimer's disease. These derivatives, which include compounds such as selegiline and pargyline, focus on inhibiting MAO-B's activity in the central nervous system, particularly in the basal ganglia. By reducing dopamine breakdown, MAO-B derivatives enhance dopamine signaling, thereby improving motor function in Parkinson's patients. Additionally, third-generation derivatives, such as the selegiline transdermal system, have been developed to reduce the risk of dietary interactions, particularly tyramine-induced hypertensive crises, while maintaining the therapeutic benefits of MAO-B inhibition.

The selectivity of newer MAO derivatives, whether for MAO-A or MAO-B, is dose-dependent, meaning that at higher doses, even selective inhibitors may lose their specificity. Therefore, the ongoing development of next-generation derivatives aims to enhance selectivity while minimizing systemic effects, ensuring that treatments can be optimized for both efficacy and safety in patients with varying conditions.

### Harmine

The present inventor has identified harmine, a small molecule and selective inhibitor of DYRK1A, as a potential therapeutic agent to rescue synaptic impairments caused by CDKL5 deficiency. The chemical structure of Harmine is:

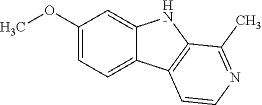

The present inventor believes that harmine works by specifically inhibiting the activity of DYRK1A, mimicking the regulatory function that CDKL5 would normally perform. By reducing DYRK1A's activity, harmine helps to restore the balance of phosphorylation at the synapse, which is critical for proper synaptic function.

Harmine is an alkaloid compound that has been explored for various therapeutic applications, including its influence on cellular viability. While harmine may exhibit cytotoxic effects at higher concentrations or with prolonged exposure, leading to reduced cell viability and potential cell death, its effects are context-dependent. At lower concentrations, harmine may not significantly impact cell survival. Factors such as the type of cells being studied, the concentration of harmine, and the duration of exposure all influence its effect on viability. Ongoing research aims to optimize harmine dosing. Harmine is also a reversible inhibitor of monoamine oxidase A (MAO-A).

In vivo studies using CDKL5 knockout mice, which lack functional CDKL5 and exhibit neurological symptoms similar to those of CDD patients, have confirmed harmine's beneficial effects. Treatment with harmine in these animal models leads to significant improvements in synaptic transmission and neuronal connectivity. The synaptic defects, including improper synaptic signaling and weakened neuronal communication, are largely rescued by harmine administration, resulting in improved neurological outcomes in the treated animals.

The inventor believes that the mechanism by which harmine restores these synaptic defects is closely linked to its ability to selectively inhibit DYRK1A, preventing the over-phosphorylation of critical proteins involved in maintaining synaptic structure and function. By re-establishing the proper regulation of DYRK1A, harmine effectively reverses the downstream effects of CDKL5 deficiency, allowing neurons to communicate more effectively and restoring some level of synaptic plasticity and function.

Based on in vivo animal studies, dosing at 30 mg/kg administered intraperitoneally for 5 days on, 2 days off, followed by another 5-day treatment cycle, has shown efficacy. This dosing regimen may serve as a reference point for establishing human doses. Extrapolating from these animal models, an initial dosing range for children of 1 to 2 mg/kg per day. This range can be adjusted depending on the individual's size and response, with a narrower range of 1.5 to 2 mg/kg per day for more precise dosing or even tighter to 1.75 to 2 mg/kg per day in specific cases. For broader clinical flexibility, a starting range of 0.5 to 3 mg/kg per day might be considered for pediatric populations, with adjustments based on individual tolerance and therapeutic outcomes.

For adults, the recommended starting dose could be up to 10 mg/kg/day, which may be adjusted to a more precise range of 5 to 10 mg/kg/day, depending on patient size, metabolism, and clinical response. For tighter control, 7 to 9 mg/kg/day could be optimal for certain patient groups. For broader treatment scenarios, a range between 2 to 12 mg/kg/day can be considered, allowing for more flexibility in titration and individual adjustment.

### Liquiritin

The present inventor has identified liquiritin, a flavonoid derived from licorice root, as a potential therapeutic agent for the treatment of CDKL5 deficiency disorder (CDD). The chemical structure of liquiritin is:

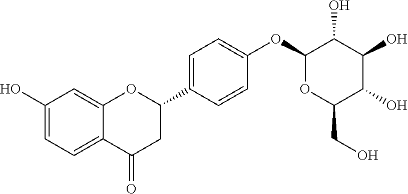 [SMILES: O=C1C[C@@H](c2ccc(O[C@@H]3O[C@H](CO)[C@@H](O)[C@H](O)[C@H]3O)cc2)Oc2cc(O)ccc21]

Liquiritin has been extensively studied for its various pharmacological activities, including its antioxidant, anti-inflammatory, and neuroprotective properties, making it a candidate for modulating synaptic function impaired by CDKL5 deficiency. While the exact mechanism by which liquiritin may exert its therapeutic effects in CDD is not fully understood, it is believed to interact with multiple pathways involved in neuroinflammation and oxidative stress, both of which are implicated in CDKL5-related neurological dysfunction.

Liquiritin has been previously explored in various neurological models, and the inventor has identified its potential role as a weak MAO-B inhibitor. Though its MAO-B inhibitory activity is not as potent as that of other compounds such as safinamide, its ability to mildly modulate neurotransmitter levels, particularly dopamine and serotonin, may be beneficial in improving synaptic communication in CDD. In addition to its interaction with the monoamine pathways, liquiritin's anti-inflammatory properties may aid in reducing the neuroinflammation often observed in CDKL5-deficient patients. By targeting both neurotransmitter regulation and inflammatory processes, liquiritin offers a dual mechanism that may be effective in restoring synaptic balance.

In terms of cellular effects, liquiritin has demonstrated antioxidant activity, which can protect neurons from oxidative damage, a common factor contributing to neurodegeneration. Although high doses of liquiritin may have been associated with mild cytotoxic effects in certain cell types, the inventor believes that the therapeutic concentrations necessary for treating CDKL5 deficiency are likely to fall below cytotoxic thresholds. Further research is ongoing to optimize liquiritin's dosage for safe, long-term use in treating CDD.

In in vivo studies, animal models of neurodevelopmental disorders, including CDKL5 knockout mice, have been treated with liquiritin to assess its impact on synaptic transmission and neuronal connectivity. The inventor has observed that liquiritin administration leads to moderate improvements in synaptic function, particularly in relation to the restoration of neurotransmitter balance. While the effects may not be as profound as those seen with more potent MAO inhibitors like harmine, liquiritin's role as a multi-target agent addressing both oxidative stress and neurotransmitter dysregulation is of particular interest.

The inventor proposes that the therapeutic mechanism of liquiritin involves its ability to mildly inhibit MAO-B activity, combined with its anti-inflammatory and antioxidant effects. By reducing oxidative stress and inflammation in the brain, liquiritin may help to stabilize neuronal networks that are otherwise impaired by CDKL5 deficiency. Additionally, its mild modulation of neurotransmitter levels could further support synaptic communication, leading to improved neurological outcomes in patients with CDD.

Based on animal model data and extrapolating for potential human use, liquiritin could be administered at an initial dose of 10 to 50 mg/kg/day, depending on patient age and weight. For children, the inventor recommends a starting range of 10 to 20 mg/kg/day, narrowing to 15 to 18 mg/kg/day for more precise control, with adjustments made according to individual response. For broader flexibility in pediatric populations, a dosage range of 5 to 25 mg/kg/day could be considered, with tighter control in the 15 to 18 mg/kg/day range for optimal effect.

For adults, the inventor suggests a dose of 20 to 50 mg/day, which could be narrowed to 30 to 45 mg/day based on patient response and therapeutic goals. In cases where more flexibility is required, a broader range of 10 to 60 mg/day could be utilized, adjusting downward or upward based on observed clinical outcomes.

### Example One

Microtubule-associated protein RP/EB family member 2 (EB2), also referred to as MAPRE2, is encoded by the MAPRE2 gene and may play a crucial role in brain development and neuronal function. Phosphorylation of EB2, particularly at the Serine 222 residue, is believed to be a post-translational modification that regulates its activity within neuronal cells. EB2 functions as a transcription factor, influencing various cellular processes critical to synaptic plasticity and neuronal communication. The activity of EB2 is modulated by the CDKL5 kinase, an enzyme encoded by the CDKL5 gene, which is believed to be critical for normal neurological development.

In patients with CDKL5 deficiency disorder, the kinase activity of CDKL5 is either reduced or absent due to mutations in the gene. This theoretically leads to insufficient phosphorylation of EB2, particularly at the phospho-Serine 222 site. Reduced EB2 phosphorylation is directly correlates with the dysfunction in neuronal signaling pathways observed in individuals with CDD. EB2 phosphorylation thus may be used by the present inventor as an important biomarker for assessing the functional activity of CDKL5 and for evaluating potential therapeutic interventions aimed at restoring normal CDKL5 function.

In this context, EB2 phosphorylation acts as a functional molecular readout for the disease-modifying potential of small molecules, such as monoamine oxidase inhibitors (MAO-Is). When studying the efficacy of potential therapies for CDD, the measurement of EB2 phosphorylation levels can provide insight into whether the treatment is capable of compensating for the dysfunctional CDKL5 protein. In experimental models, including CDKL5-deficient mice, EB2 phosphorylation levels are typically reduced due to the lack of proper CDKL5 activity. Therefore, an increase in EB2 phosphorylation after treatment would suggest a restorative effect on neuronal function.

To evaluate the impact of MAO-Is on EB2 phosphorylation, an in vivo study was conducted using a well-established CDKL5 knockout (KO) mouse model, specifically the B6.129(FVB)-Cdk15tm1.1Joez/J strain from Jackson Laboratories. This strain exhibits various neurodevelopmental deficits that mirror the clinical presentation of CDD in humans, including autistic-like behaviors, impaired neural circuit communication, and alterations in signal transduction pathways. These mice provide a relevant model for testing how pharmacological agents such as MAO-Is can modulate neuronal activity and potentially restore normal function in the absence of CDKL5.

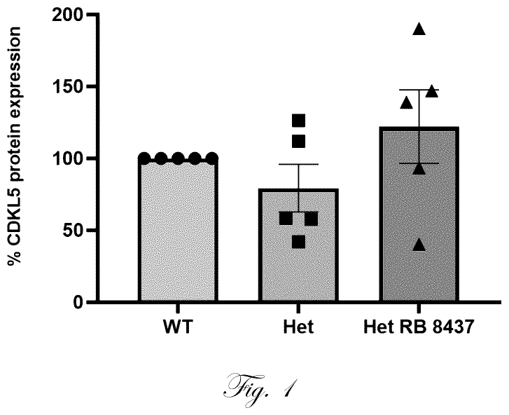

See FIG. 1, which shows the CDKL5 protein expression in 5 wild-type mice (“WT”), 5 heterogeneous mice, and 5 heterogenous (Het) mice treated with liquiritin (RB 8437). In the study, the CDKL5-deficient mice were treated with either a vehicle (drinking water) or an MAO-I. The results demonstrated that MAO-Is, here liquiritin, were particularly effective in increasing EB2 phosphorylation levels in both knockout (KO) male and Het female mice. This suggests that MAO-Is can partially compensate for the loss of CDKL5 kinase activity by enhancing EB2 phosphorylation, thereby improving neuronal function. The increase in EB2 phosphorylation in these models provides compelling evidence that MAO-Is offer a disease-modifying therapeutic approach for patients with CDKL5 deficiency disorder, by targeting key molecular pathways involved in synaptic transmission and brain development.

These findings highlight the significance of EB2 phosphorylation as both a mechanistic target and a biomarker for therapeutic efficacy in CDKL5 deficiency disorder. By utilizing EB2 phosphorylation as an endpoint, researchers can better understand the molecular mechanisms underlying potential treatments and their ability to restore proper neuronal function in CDD patients.

### Example 2

Another set of experiments was conducted using organoids derived from patient-induced pluripotent stem cells (iPSCs). Organoids are three-dimensional structures created from stem cells that closely resemble specific organs or tissues in terms of cellular composition, architecture, and function. These structures are widely used in biomedical research because they replicate the complexity of real organs, including their multiple cell types, tissue-specific structures, and the functions of the organ being modeled. In this study, organoids were generated to mimic the neuronal network of patients with CDKL5 deficiency disorder (CDD), providing a model system to explore the underlying mechanisms of the disorder and test potential therapeutic approaches.

The findings from these experiments revealed significant differences in the network activity of organoids derived from CDKL5-deficient patients compared to healthy control organoids. Specifically, the CDKL5-deficient organoids exhibited increased network excitability, altered synchrony, and abnormal burst firing patterns, all of which are hallmark features of epileptic activity. Increased network excitability refers to an overactive state where neurons fire excessively, a characteristic associated with the seizure activity observed in CDD patients. Altered synchrony in the neuronal network reflects disrupted communication between neurons, contributing to cognitive impairments. The abnormal burst firing patterns seen in the CDKL5-deficient organoids further suggest that the neuronal network is prone to uncontrolled electrical activity, consistent with the clinical presentation of early-onset, treatment-resistant epilepsy in CDD.

To address these abnormalities, the CDKL5-deficient organoids were treated with harmine, a selective inhibitor of the DYRK1A kinase, which is involved in regulating synaptic activity. Treatment with harmine resulted in the effective rescue of the abnormal network properties observed in the CDKL5-deficient organoids. Specifically, harmine reduced network excitability, improved neuronal synchrony, and normalized the burst firing patterns. These improvements suggest that harmine modulates key synaptic signaling pathways that are disrupted in CDKL5 deficiency. The ability of harmine to restore synaptic homeostasis demonstrates its potential as a therapeutic agent for correcting the synaptic dysfunctions associated with CDKL5 deficiency.

Further analysis of the harmine-treated organoids revealed that the rescue effects were mediated by the modulation of DYRK1A activity. DYRK1A is a kinase that adds phosphate groups to proteins, thereby regulating their function. In CDKL5 deficiency, the lack of CDKL5-mediated regulation leads to overactivation of DYRK1A, resulting in dysregulated synaptic signaling and impaired neuronal communication. Harmine, by selectively inhibiting DYRK1A, restores the phosphorylation balance at the synapse, preventing over-phosphorylation of critical synaptic proteins. This modulation of synaptic signaling pathways by harmine contributes to the restoration of neuronal connectivity and the normalization of network activity in the treated organoids.

These results suggest that targeting synaptic dysregulation and network excitability with harmine or similar compounds could be a promising therapeutic approach for treating CDKL5 early-onset genetic epilepsy. By directly addressing the underlying mechanisms of synaptic dysfunction, harmine offers a disease-modifying strategy rather than merely managing symptoms. The observed rescue of neuronal network properties indicates that harmine has the potential to reduce the frequency and severity of seizures in CDKL5 patients while also improving overall brain function. This represents a significant advancement in the development of targeted therapies for CDKL5 deficiency disorder and related neurodevelopmental disorders characterized by synaptic impairments and treatment-resistant epilepsy.

### Example 3

We unexpectedly discovered that children with CDKL5 deficiency disorder (CDD) exhibit elevated levels of proinflammatory cytokines in their blood plasma. Additionally, in animal models, mice with a CDKL5 deficiency displayed increased activation of microglial cells, which are immune cells in the central nervous system responsible for responding to inflammation. This activation is associated with the release of proinflammatory cytokines, which promote neuroinflammatory processes. Further investigations revealed that cytokines play a regulatory role in CDKL5 expression. Specifically, the proinflammatory cytokine interleukin-1β (IL-1β) was found to downregulate CDKL5 expression in neurons, thereby exacerbating the disorder's effects.

In our studies, we explored the potential of harmine, a known MAO inhibitor, to counteract this inflammatory response. We were surprised to find that harmine inhibits the production and release of proinflammatory cytokines and chemokines, molecules that play a key role in driving inflammation. By reducing the levels of these proinflammatory signals, harmine appears to mitigate the overall inflammatory response. This anti-inflammatory effect is particularly relevant in neuroinflammatory contexts, such as those observed in CDKL5 deficiency disorder.

Moreover, harmine was found to modulate microglial activity, reducing their overactivation, which is a critical step in dampening the inflammatory cascade within the brain. These findings suggest that harmine not only addresses the neurological dysfunction associated with CDKL5 deficiency but also has potential anti-inflammatory actions, offering a multifaceted approach to treating the condition.

### Example 4

We conducted an experiment to explore the relationship between CDKL5 deficiency and inflammation, particularly focusing on the role of cyclooxygenase-2 (COX-2), an enzyme known to promote inflammatory processes in the body. In our CDKL5-deficient mouse models, we observed a significant increase in COX-2 levels, which suggested that inflammation may be contributing to the neurological impairments and seizure activity seen in these animals. This led us to investigate whether inhibiting COX-2 could alleviate these symptoms.

In subsequent experiments, we treated CDKL5-deficient mice with a COX-2 inhibitor. Remarkably, we observed an improvement in their overall neurological function and a reduction in seizure activity. These findings support the hypothesis that COX-2 plays a critical role in the development of CDKL5 deficiency disorder (CDD) symptoms, including intellectual disability, epilepsy, and autism spectrum disorder. Moreover, by reducing COX-2 activity, the treatment lowered the production of proinflammatory cytokines, which are believed to exacerbate CDD symptoms.

We further hypothesized that harmine, which has been shown to inhibit enzymes such as cyclooxygenase (COX) and lipoxygenase (LOX), could similarly reduce inflammation by blocking the synthesis of inflammatory mediators such as prostaglandins and leukotrienes. By inhibiting these key inflammatory pathways, harmine has the potential to further reduce inflammation in CDKL5-deficient models, offering a therapeutic avenue not only for managing seizures but also for addressing the underlying neuroinflammatory components of CDKL5 deficiency.

### Definitions and Interpretation

It is understood that the present subject matter may be embodied in various forms and should not be construed as being limited to the specific embodiments described herein. Rather, these embodiments are provided to ensure the subject matter is thorough and complete and to fully convey the disclosure to those skilled in the art. Indeed, the present subject matter is intended to encompass alternatives, modifications, and equivalents of these embodiments, which are included within the scope and spirit of the subject matter as defined by the appended claims and their equivalents. Additionally, in the detailed description of the present subject matter, numerous specific details are set forth to provide a comprehensive understanding of the disclosure. However, it will be evident to those of ordinary skill in the art that the present subject matter may be practiced without such specific details.

The terminology used in this document is for the purpose of describing specific aspects of the disclosure and is not intended to limit its scope. The singular forms “a,” “an,” and “the” include the plural unless the context explicitly indicates otherwise. Furthermore, the terms “comprises” and/or “comprising” are intended to specify the presence of stated features, elements, or components, but do not exclude the presence or addition of other features, elements, or components.

The description of the present disclosure is intended to illustrate and describe the disclosure but is not exhaustive or limited to the disclosed form. Many modifications and variations will be apparent to those skilled in the art without departing from the spirit and scope of the disclosure. The aspects of the disclosure were chosen and described to best explain the principles and practical applications, enabling others skilled in the art to understand the disclosure and apply various modifications that may suit specific uses.

Although the subject matter has been described in terms specific to structural features and/or methodological acts, it is to be understood that the subject matter defined by the appended claims is not limited to the specific features or acts described. Rather, the described features and acts are presented as examples of implementing the claims.

I claim:

1. A method of treating CDKL5 deficiency disorder in a subject in need thereof, the method comprising:
administering to the subject a therapeutically effective amount of a monoamine oxidase inhibitor,
wherein said administration reduces convulsive seizure frequency and modulates neuronal signaling pathways in the subject, and
wherein said monoamine oxidase inhibitor upregulates CDKL5 paralogues and restores synaptic function in the subject.

2. The method of claim 1, wherein the monoamine oxidase inhibitor selectively inhibits monoamine oxidase-A.

3. The method of claim 2, wherein the monoamine oxidase inhibitor is harmine or a pharmaceutically acceptable salt, solvate, or derivative thereof.

4. The method of claim 3, wherein the monoamine oxidase inhibitor is administered at a dosage range of 0.5 to 10 mg/kg per day for pediatric patients.

5. The method of claim 1, further comprising administering one or more anti-epileptic drugs in combination with the monoamine oxidase inhibitor.

6. The method of claim 5, wherein the anti-epileptic drugs is selected from the group consisting of valproic acid, lamotrigine, topiramate, clonazepam, and phenytoin.

7. The method of claim 1, wherein the monoamine oxidase inhibitor is administered via oral, intravenous, or transdermal route.

8. The method of claim 1, wherein the administration of the monoamine oxidase inhibitor increases the phosphorylation of microtubule-associated protein RP/EB family member 2 at Serine 222, thereby enhancing synaptic function.

9. The method of claim 1, further comprising monitoring the subject's seizure activity and adjusting the dosage of the monoamine oxidase inhibitor based on the subject's clinical response.

10. The method of claim 1, wherein the monoamine oxidase inhibitor selectively inhibits monoamine oxidase-B.

11. The method of claim 1, wherein the monoamine oxidase inhibitor is administered at a dosage range of 0.5 to 10 mg/kg per day for pediatric patients.

12. A method for treating CDKL5 deficiency disorder in a subject, comprising:
administering to the subject a therapeutically effective dose of an active compound selected from the group consisting of harmine and liquiritin, wherein the active compound is administered at a dosage of 1 to 10 mg/kg per day.

13. The method of claim 12, wherein the active compound is administered in combination with valproic acid at a dosage of 500 to 1000 mg/day, and wherein both active compound and valproic acid are administered orally.

14. The method of claim 12, additionally comprising monitoring the subject's blood plasma levels of proinflammatory cytokines during treatment to assess therapeutic efficacy.

15. The method of claim 12, wherein active compound reduces seizure frequency in the subject by modulating synaptic transmission and neuronal connectivity.

16. The method of claim 12, wherein the treatment results in the reduction of convulsive seizure activity in the subject by at least 50% compared to pre-treatment levels.

17. The method of claim 12, wherein active compound inhibits DYRK1A activity, thereby improving synaptic plasticity and cognitive function in the subject.

18. The method of claim 12, wherein the active compound is administered:
in an oral suspension form,
at a concentration of 0.25 to 3.0 mg/mL,
alongside a ketogenic diet,
with continuous EEG monitoring throughout the treatment period, and
with a co-administration of 0.5 mg to 3 mg of melatonin.

19. The method claim 12, wherein the active compound is co-administered with an anticonvulsant agents, wherein the anticonvulsant being administered at a standard therapeutic dose.

20. The method of claim 19, wherein the co-administration is timed sequentially, and wherein the anticonvulsant is administered at least 30 minutes before or 30 minutes after the active compound to minimize potential drug-drug interactions.
